(19) United States
(12) Patent Application Publication
(10) Pub. No.: US 2025/0319242 A1
(43) Pub. Date: Oct. 16, 2025

(54) SURGICAL DRAIN SYSTEM

(71) Applicant: Darlene Frances Harmon, Morgan Hill, CA (US)

(72) Inventor: Darlene Frances Harmon, Morgan Hill, CA (US)

(21) Appl. No.: 18/636,323

(22) Filed: Apr. 16, 2024

(51) Int. Cl. A61M 1/00

(52) U.S. Cl. CPC A61M 1/912; A61M 2205/583; A61M 2205/587

(57) ABSTRACT

A surgical drain system includes an adherent flange, a drainage tubing, and a suction drainage container. The adherent flange is attached to a skin surface of a patient's body, via a washer, external to an incision site, thereby securing the surgical drain system to the body, free of sutures. The drainage tubing includes a flexible corrugated section connected in fluid communication between two flexible drain sections. A proximal end of a first flexible drain section is inserted into the incision site, while the flexible corrugated section and a second flexible drain section are disposed external to the incision site. The suction drainage container suctions and collects fluid from a wound or surgical site through the drainage tubing. A flexible, cone-shaped, hollow member is inserted into and disposed inside the incision site to preclude the proximal end of the first flexible drain section from being accidentally dislodged from the incision site.

### BACKGROUND

When an initial incision is made at the beginning of a surgical procedure, a wound or a surgical site is formed, into which a patient's body conveys, for example, blood, blood products, proteins, etc. Tissues that are cut during the surgical procedure tend to leak fluids comprising, for example, wound exudate, blood cells, blood products, blood clots, wound debris, dead cells, pus, serous fluid, and other byproducts of wound healing. Removal of these fluids is required to promote healing of the tissues to reduce the chance of infection and to reduce discomfort which may be caused by increased pressure within an incision site. If the fluids are not removed properly, the fluids accumulate under a patient's skin and cause problems.

A surgical drain is typically used to drain fluids that accumulate in the wound or the surgical site under the patient's skin, proximal to an incision site. An example of a surgical drain is the Jackson-Pratt (JP) drain. The Jackson-Pratt drain is a closed suction device where fluids are collected within a closed system, without an external suction machine. The Jackson-Pratt drain comprises a thin, flexible rubber tube, one end of which enters through the incision site and sits under a patient's skin in a region under or proximal to the incision site. The skin and one end of the tube are sutured or stitched together to preclude the Jackson-Pratt drain from moving and accidentally being pulled out or dislodged from under the skin. A bulb is connected to the other end of the tube for suctioning and collecting the fluids from the wound or the surgical site through the tube. When the bulb is squeezed, air is removed from the bulb, thereby creating a negative pressure or a suctioning effect that draws the fluids out from the wound or the surgical site and into the bulb. The bulb expands as fluid fills therein over time.

In an example, to fix a surgical drain at the incision site, two non-resorbable surgical sutures are typically placed at the incision site. The surgical sutures pierce the skin and are tied around the circumference of the surgical drain. These surgical sutures typically irritate the patient's skin, cause pain at the incision site, and may cause infection. Moreover, when the patient moves about, the surgical drain may get caught on something, which may tear the sutures from the skin and allow the proximal end of the tube of the surgical drain to be dislodged from the incision site, resulting in an accumulation of fluids at the incision site that would delay or adversely affect the wound healing process. Furthermore, in some cases, the proximal end of the tube of the surgical drain may be incorrectly positioned at the incision site or at the wound or the surgical site, causing improper drainage of the fluids therefrom.

Hence, there is a long-felt need for a surgical drain system comprising a device that secures the surgical drain system to a body of a patient, free of sutures, while precluding a proximal end of a drainage tubing from being accidentally dislodged from the incision site. Furthermore, there is a need for an apparatus that allows proper positioning of the proximal end of the drainage tubing of the surgical drain system in the incision site and further into the wound or the surgical site to allow proper drainage of the fluids from the wound or the surgical site.

### BRIEF DESCRIPTION OF THE DRAWINGS

The following detailed description of the invention is better understood when read in conjunction with the appended drawings. For illustrating the embodiments herein, exemplary constructions of the embodiments are shown in the drawings. However, the embodiments herein are not limited to the specific components, structures, and methods disclosed herein. In an embodiment, various structural elements are employed depending on design choices of a system designer. The description of a component, or a structure, or a method step referenced by a numeral in a drawing is applicable to the description of that component, or structure, or method step shown by that same numeral in any subsequent drawing herein. The terms “right”, “left”, “side”, “front”, “rear”, “top”, “bottom”, etc., are based on an orientation or a positional relationship shown in the appended drawings, and are recited merely for describing the embodiments herein, rather than indicating or implying that the device, component, or structure referenced must have a particular orientation or position or must be constructed and operated in a particular orientation, and therefore should not be construed as limiting the embodiments herein.

FIG. 1A illustrates an assembled, right-side perspective view of an embodiment of a surgical drain system.

FIG. 1B illustrates a disassembled, right-side perspective view of the embodiment of the surgical drain system shown in FIG. 1A.

FIG. 1C illustrates a right-side elevation view of the embodiment of the surgical drain system shown in FIG. 1A, showing a proximal end of a drainage tubing of the surgical drain system inserted into an incision site under a skin surface.

FIG. 1D illustrates a left-side elevation view of the embodiment of the surgical drain system shown in FIG. 1A.

FIG. 1E illustrates a front elevation view of the embodiment of the surgical drain system shown in FIG. 1A.

FIG. 1F illustrates a rear elevation view of the embodiment of the surgical drain system shown in FIG. 1A.

FIG. 1G illustrates a top plan view of the embodiment of the surgical drain system shown in FIG. 1A.

FIG. 1H illustrates a bottom elevation view of the embodiment of the surgical drain system shown in FIG. 1A.

FIG. 2A illustrates an assembled, right-side perspective view of another embodiment of the surgical drain system.

FIG. 2B illustrates a disassembled, right-side perspective view of the embodiment of the surgical drain system shown in FIG. 2A.

FIG. 2C illustrates a right-side elevation view of the embodiment of the surgical drain system shown in FIG. 2A, showing a proximal end of a drainage tubing of the surgical drain system inserted into an incision site under a skin surface.

FIG. 2D illustrates a left-side elevation view of the embodiment of the surgical drain system shown in FIG. 2A.

FIG. 2E illustrates a top plan view of the embodiment of the surgical drain system shown in FIG. 2A.

FIG. 2F illustrates a bottom elevation view of the embodiment of the surgical drain system shown in FIG. 2A.

FIG. 2G illustrates a front elevation view of the embodiment of the surgical drain system shown in FIG. 2A.

FIG. 2H illustrates a rear elevation view of the embodiment of the surgical drain system shown in FIG. 2A.

FIG. 2I illustrates a partial sectional view of the embodiment of the surgical drain system shown in FIG. 2A.

FIGS. 3A-3C illustrate right-side perspective views of an embodiment of a retractable sheath configured to allow proper positioning of a proximal end of a drainage tubing of the surgical drain system in an incision site.

FIG. 4 illustrates a right-side perspective view showing positioning of the retractable sheath in an incision site.

FIG. 5 illustrates a right-side perspective view showing positioning of the proximal end of the drainage tubing of the surgical drain system through the retractable sheath.

FIG. 6A illustrates an assembled, right-side perspective view of another embodiment of the surgical drain system.

FIG. 6B illustrates a right-side elevation view of the embodiment of the surgical drain system shown in FIG. 6A, showing a proximal end of a drainage tubing of the surgical drain system inserted into an incision site under a skin surface.

FIG. 6C illustrates a front elevation view of the embodiment of the surgical drain system shown in FIG. 6A.

FIG. 6D illustrates a cross-sectional view of the embodiment of the surgical drain system shown in FIG. 6A, taken along a sectional line A-A shown in FIG. 6C.

FIG. 6E illustrates a right-side elevation view of the embodiment of the surgical drain system shown in FIG. 6A, showing a distal end of the drainage tubing of the surgical drain system connected to a suction drainage container.

FIG. 6F illustrates a right-side elevation view of the embodiment of the surgical drain system shown in FIG. 6A, showing another embodiment of the drainage tubing of the surgical drain system.

FIG. 7A illustrates an assembled, right-side perspective view of another embodiment of the surgical drain system.

FIG. 7B illustrates a right-side elevation view of the embodiment of the surgical drain system shown in FIG. 7A, showing a proximal end of a drainage tubing of the surgical drain system inserted into an incision site under a skin surface.

FIG. 7C illustrates a front elevation view of the embodiment of the surgical drain system shown in FIG. 7A.

FIG. 7D illustrates a cross-sectional view of the embodiment of the surgical drain system shown in FIG. 7A, taken along a sectional line B-B shown in FIG. 7C.

FIG. 7E illustrates a right-side elevation view of the embodiment of the surgical drain system shown in FIG. 7A, showing a distal end of the drainage tubing of the surgical drain system connected to a suction drainage container.

FIG. 7F illustrates a right-side elevation view of the embodiment of the surgical drain system shown in FIG. 7A, showing another embodiment of the drainage tubing of the surgical drain system.

### DETAILED DESCRIPTION OF THE INVENTION

The system disclosed herein addresses the above-recited need for a surgical drain system comprising a device that secures the surgical drain system to a body of a patient, free of sutures, while precluding a proximal end of a drainage tubing from being accidentally dislodged from an incision site. Furthermore, the system disclosed herein addresses the above-recited need for an apparatus that allows proper positioning of the proximal end of the drainage tubing of the surgical drain system in the incision site, and further into a wound or a surgical site to allow proper drainage of fluids from the wound or the surgical site. As used herein, “incision site” refers to an area on the patient's body where a surgical cut or opening is made for executing a surgical procedure. The size, location, and number of incisions made in the patient's body can vary depending on the type of surgery. The proximal end of the drainage tubing of the surgical drain system enters the patient's body through the incision site and sits under the patient's skin in a region under or proximal to the incision site. Also, as used herein, “the wound or the surgical site” refers to a site in the patient's body where a surgical procedure has been performed and into which the patient's body conveys, for example, blood, blood products, bone fragments, proteins, etc.

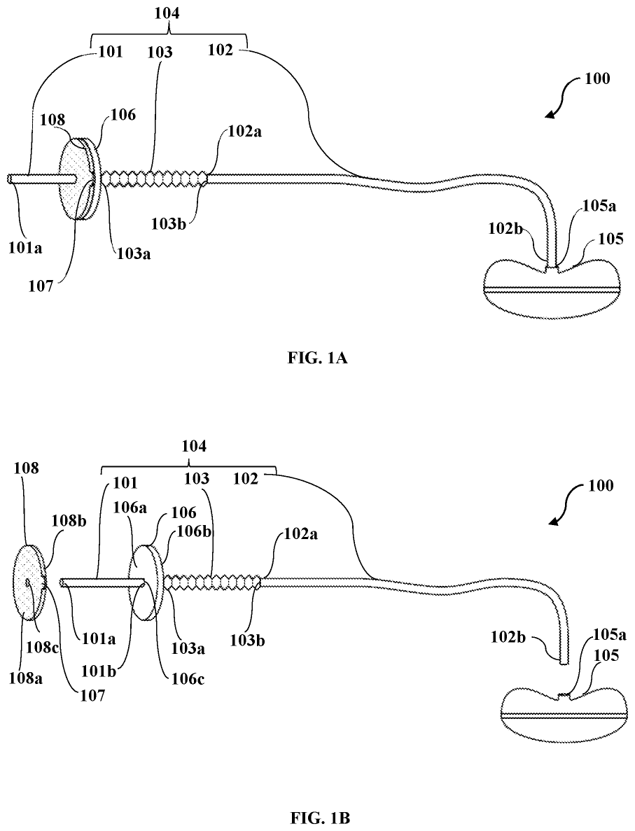

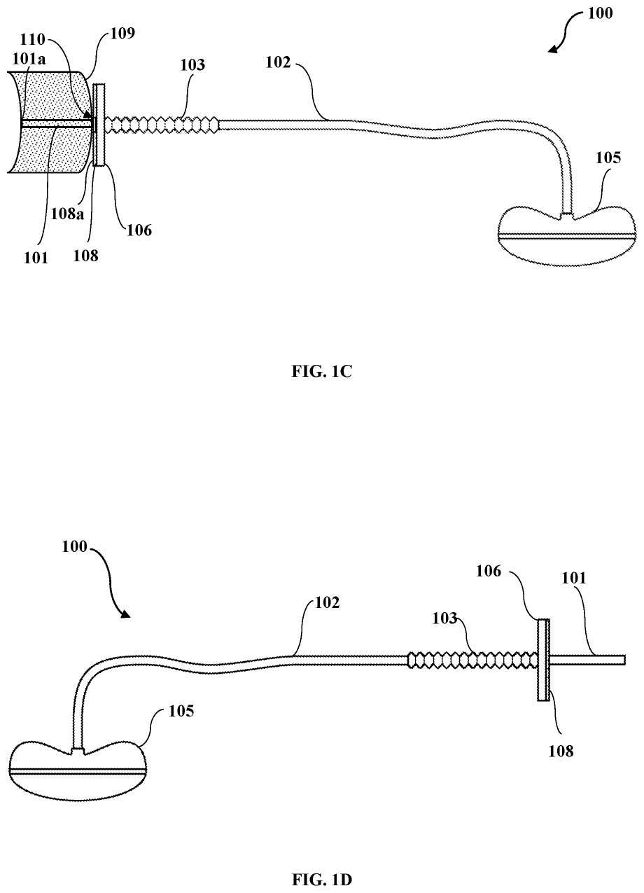

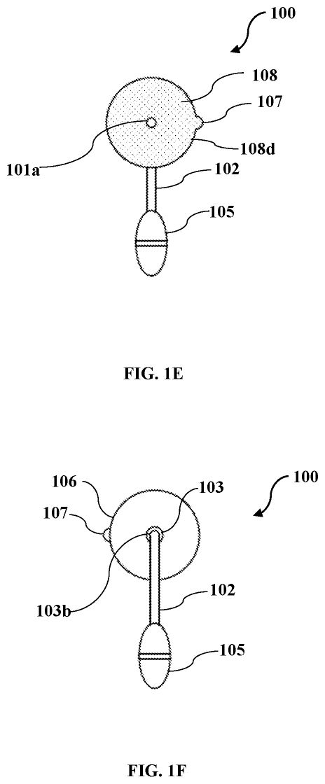

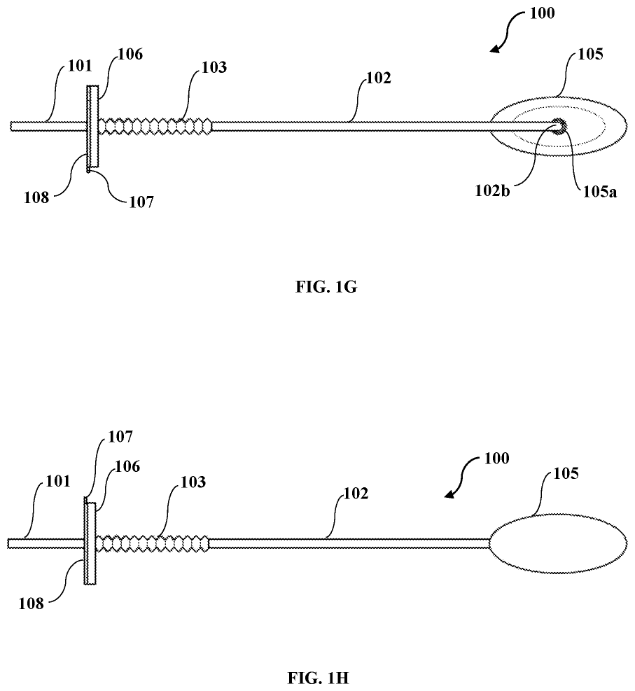

FIGS. 1A-1B illustrate an assembled, right-side perspective view and a disassembled, right-side perspective view of an embodiment of a surgical drain system 100, respectively. The surgical drain system 100 comprises an adherent flange 106, a washer 108, a drainage tubing 104, and a suction drainage container 105. The adherent flange 106 is configured to secure the surgical drain system 100 to a body of a patient, free of sutures, via the washer 108. The adherent flange 106 is, for example, of a generally circular shape. The adherent flange 106 comprises a first surface 106a, a second surface 106b, and a central opening 106c as illustrated in FIG. 1B. The terms “first” and “second” are used herein for descriptive purposes only and are not to be construed to indicate or imply relative importance. The adherent flange 106 is made from a thin flexible material, for example, molded plastic. In an example, the diameter and the thickness of the adherent flange 106 is about 6 inches and about 0.125 inch, respectively.

In an embodiment, the adherent flange 106 is configured to be attached to a skin surface 109 of the body via the washer 108 and be disposed external to an incision site 110 of the body as illustrated in FIG. 1C. In an embodiment as illustrated in FIGS. 1A-1B, the washer 108 is detachably attachable and disposed coaxial to the adherent flange 106 between the skin surface 109 and the adherent flange 106. The washer 108 attaches to the skin surface 109. The adherence of the washer 108 to the skin surface 109 allows the adherent flange 106 and the drainage tubing 104 to be removed after a few weeks of draining. The washer 108 that is attached to the skin surface 109, for example, by surgical glue or by its adhesive surface, can be removed safely when the surgical glue breaks down, for example, after three or four weeks. The washer 108 is, for example, of a generally circular shape.

In an embodiment as illustrated in FIG. 1B, the washer 108 comprises a first surface 108a, a second surface 108b, and a central opening 108c. The first surface 108a of the washer 108 is configured to be attached flush against the skin surface 109 as illustrated in FIG. 1C. The first surface 106a of the adherent flange 106 is configured to be attached flush against the second surface 108b of the washer 108. Examples of adhesives used on the first surface 106a of the adherent flange 106 to attach the first surface 106a of the adherent flange 106 to the second surface 108b of the washer 108 are removable medical adhesives, 3M® adhesives, etc. In an embodiment, the first surface 106a of the adherent flange 106 comprises a peel and stick adhesive component that can be peeled and attached to the second surface 108b of the washer 108. A first flexible drain section 101 of the drainage tubing 104 is inserted through the central openings 106c and 108c of the adherent flange 106 and the washer 108, respectively, for insertion into the incision site 110. In an embodiment, the first surface 108a of the washer 108 is an adhesive surface configured to be attached directly to the skin surface 109. In another embodiment, the first surface 108a of the washer 108 is a smooth surface configured to be attached to the skin surface 109 using surgical glue. Examples of adhesives used on the first surface 108a of the washer 108 to attach the first surface 108a of the washer 108 to the skin surface 109 are removable medical adhesives, 3M® adhesives, etc. In an example, the diameter and the thickness of the washer 108 are about 6 inches and about 0.0625 inches, respectively. The washer 108 is made, for example, from silicone.

In an embodiment as illustrated in FIGS. 1A-1B and FIG. 1E, the surgical drain system 100 further comprises a tab 107 extending from a periphery 108d of the washer 108. The tab 107 is configured to allow removal of the surgical drain system 100 from the incision site 110. A user may hold the tab 107 between two fingers and pull out the first flexible drain section 101 of the drainage tubing 104 along with the adherent flange 106 from the incision site 110. The adherent flange 106 with the drainage tubing 104 may be detached from the washer 108 that is attached to the skin surface 109, and removed from the body after a predetermined time period, for example, about 14 days, while leaving the washer 108 on the skin surface 109 until the adhesive or the surgical glue comes off from the body. In an embodiment, when the adherent flange 106 with the drainage tubing 104 is removed from the body, the washer 108 is attached to and left on the skin surface 109, for example, using an adhesive bandage, to cover the wound. The adhesive bandage is disposed on the washer 108 externally on the skin surface 109.

In an embodiment, the adherent flange 106 is configured to be attached to the skin surface 109 of the body directly without the washer 108, via the first surface 106a of the adherent flange 106, and be disposed external to the incision site 110 of the body. In an embodiment, the first surface 106a of the adherent flange 106 is an adhesive surface configured to be attached directly to the skin surface 109. In another embodiment, the first surface 106a of the adherent flange 106 is a smooth surface configured to be attached to the skin surface 109 using surgical glue. The adhesive surface of the adherent flange 106 or the surgical glue on the first surface 106a of the adherent flange 106 is used to adhere the adherent flange 106 to the skin surface 109 to fix a proximal end 101a of the drainage tubing 104 to the incision site 110. Examples of adhesives used on the first surface 106a of the adherent flange 106 to attach the first surface 106a of the adherent flange 106 to the skin surface 109 are removable medical adhesives, 3M® adhesives, etc.

The drainage tubing 104 is operably coupled to the adherent flange 106 and the washer 108. In an embodiment, the adherent flange 106 is pre-molded to the drainage tubing 104. The length of the drainage tubing 104 is, for example, about 16 inches. In an embodiment as illustrated in FIGS. 1A-1B, the drainage tubing 104 is configured as a single continuous tube comprising three sections, that is, a first flexible drain section 101, a second flexible drain section 102, and a flexible corrugated section 103. The first flexible drain section 101, the second flexible drain section 102, and the flexible corrugated section 103 of the drainage tubing 104 are made, for example, from plastic, flexible rubber, latex, silicone, polyethylene, polyvinyl chloride, silicone elastomers, etc. The first flexible drain section 101 of the drainage tubing 104 is inserted through the central openings 106c and 108c of the adherent flange 106 and the washer 108, respectively. The first flexible drain section 101 comprises a proximal end 101a and a distal end 101b as illustrated in FIG. 1B. The proximal end 101a of the first flexible drain section 101 is configured to be inserted into the incision site 110 under the skin surface 109 and positioned further into a wound or a surgical site (not shown) in the patient's body to receive fluid, for example, wound exudate, blood cells, blood products, blood clots, wound debris, dead cells, pus, serous fluid, etc., from the wound or the surgical site. In an embodiment, the adherent flange 106 is movable over the first flexible drain section 101 to accommodate a required length of the first flexible drain section 101 during insertion into the incision site 110. The drainage tubing 104 is configured to receive and pass the fluid from the wound or the surgical site into the suction drainage container 105 through the first flexible drain section 101. The fluid received through the first flexible drain section 101 of the drainage tubing 104 is suctioned into and collected in the suction drainage container 105. In an example, the length of the first flexible drain section 101 of the drainage tubing 104 is about 3 inches to about 4 inches and can be cut to any required size.

The flexible corrugated section 103 of the drainage tubing 104 comprises a first end 103a and a second end 103b as illustrated in FIGS. 1A-1B. The first end 103a of the flexible corrugated section 103 is connected to the distal end 101b of the first flexible drain section 101 and is disposed external to the incision site 110. The flexible corrugated section 103 is in fluid communication with the first flexible drain section 101. In an example, the length of the flexible corrugated section 103 is about 2 inches to about 3 inches. In an example, the diameter of the flexible corrugated section 103 ranges from about 0.15 inches to about 0.3 inches. The first end 103a of the flexible corrugated section 103 is configured to lie flush against the second surface 106b of the adherent flange 106. In an embodiment, the second surface 106b of the adherent flange 106 is molded to the first end 103a of the flexible corrugated section 103. The flexible corrugated section 103 is configured to reduce pain and discomfort experienced by the patient due to accidental movement of the first flexible drain section 101 and the second flexible drain section 102. As opposed to a straight tube that causes substantial pain to the patient during movement of the straight tube, the flexible corrugated section 103 moves along with the body and reduces pain. The flexible corrugated section 103 expands and contracts along with movement of the body, thereby reducing pain and discomfort experienced by the patient.

The second flexible drain section 102 comprises a first end 102a and a second end 102b as illustrated in FIGS. 1A-1B. The first end 102a of the second flexible drain section 102 is connected to the second end 103b of the flexible corrugated section 103. The second flexible drain section 102 is in fluid communication with the flexible corrugated section 103. In an example, the length of the second flexible drain section 102 is about 12 inches to about 14 inches. In another example, the length of the second flexible drain section 102 is about 7 inches to about 9 inches. In another example, the total length of the drainage tubing 104 from the proximal end 101a of the first flexible drain section 101 to the second end 102b of the second flexible drain section 102 is about 12 inches to about 14 inches. The drainage tubing 104 is configured as a single piece from the proximal end 101a of the first flexible drain section 101 to the second end 102b of the second flexible drain section 102. The drainage tubing 104 is manufactured by processing medical-grade materials, for example, through extrusion or molding. During an extrusion process, a medical-grade material is forced through a die to create a continuous profile with a desired cross-sectional shape of the drainage tubing 104. During a molding process, the medical-grade material is injected into a mold cavity to produce the specific shape of the drainage tubing 104. When the medical-grade material is formed into the required shape, the drainage tubing 104 is cut or trimmed to a desired length and shape.

The suction drainage container 105 is detachably connected to the second end 102b of the second flexible drain section 102 of the drainage tubing 104. In an embodiment, the suction drainage container 105 is of a generally ovular shape as illustrated in FIGS. 1A-1H. In an example, the length and the width of the suction drainage container 105 is about 3 inches and about 3.5 inches, respectively. The suction drainage container 105 is made, for example, from a silicone material, a plastic material, etc. The suction drainage container 105 comprises a port 105a for inserting and connecting the second end 102b of the second flexible drain section 102 to the suction drainage container 105. The suction drainage container 105 is configured to create a negative pressure therewithin to suction and collect the fluid received from the wound or the surgical site through the drainage tubing 104. In an embodiment, the suction drainage container 105 is configured as a squeeze bulb, which when squeezed, deflates and removes air out from the suction drainage container 105, thereby creating a negative pressure or a suctioning effect that draws the fluid out from the wound or the surgical site and into the suction drainage container 105. The suction drainage container 105 expands as the fluid fills therein over time.

FIGS. 1C-1D illustrate a right-side elevation view and a left-side elevation view of the embodiment of the surgical drain system 100 shown in FIG. 1A, respectively. FIG. 1C illustrates the proximal end 101a of the drainage tubing 104 of the surgical drain system 100 inserted into an incision site 110 of a patient's body under a skin surface 109. FIG. 1C also illustrates the first surface 108a of the washer 108 lying flush against the skin surface 109 of the patient's body, when the proximal end 101a of the first flexible drain section 101 is inserted into the incision site 110 of the patient's body. The washer 108 and the adherent flange 106 are disposed external to the incision site 110. In an example, the length of the part of the drainage tubing 104 inserted under the skin surface 109, that is, the first flexible drain section 101, is about 3 inches, and the flexible corrugated section 103 extends to about 2 inches from the molded adherent flange 106. The adherent flange 106 is molded on the first flexible drain section 101 before the flexible corrugated section 103.

FIGS. 1E-1F illustrate a front elevation view and a rear elevation view of the embodiment of the surgical drain system 100 shown in FIG. 1A, respectively. The front elevation view in FIG. 1E illustrates the proximal end 101a of the first flexible drain section 101 extending through the coaxial openings 108c and 106c of the washer 108 and the adherent flange 106, respectively, illustrated in FIG. 1B. The rear elevation view in FIG. 1F illustrates the second flexible drain section 102 extending from the second end 103b of the flexible corrugated section 103 of the drainage tubing 104 illustrated in FIGS. 1A-1B. FIGS. 1E-1F also illustrate the tab 107 extending from the periphery 108d of the washer 108.

FIGS. 1G-1H illustrate a top plan view and a bottom elevation view of the embodiment of the surgical drain system 100 shown in FIG. 1A, respectively. The top plan view in FIG. 1G illustrates the second end 102b of the second flexible drain section 102 connected to the port 105a of the suction drainage container 105.

Consider an example where a wound of a patient needs to be drained using the surgical drain system 100 comprising the washer 108, the adherent flange 106, the drainage tubing 104, and the suction drainage container 105 illustrated in FIGS. 1A-1H. The drainage tubing 104 is operably coupled to the adherent flange 106 as illustrated in FIG. 1B. That is, the first flexible drain section 101 of the drainage tubing 104 passes through the central opening 106c of the adherent flange 106 such that the distal end 101b of the first flexible drain section 101 connects to the first end 103a of the flexible corrugated section 103 through the central opening 106c of the adherent flange 106, and the first end 103a of the flexible corrugated section 103 lies flush against the second surface 106b of the adherent flange 106. A user, for example, a nurse practitioner, slips the washer 108 onto the first flexible drain section 101 of the drainage tubing 104 through its central opening 108c and attaches the second surface 108b of the washer 108 to the first surface 106a of the adherent flange 106 illustrated in FIG. 1B, using surgical glue, before inserting the proximal end 101a of the first flexible drain section 101 into the patient's body. In an embodiment, the user peels a peel and stick adhesive component provided on the first surface 106a of the adherent flange 106 and attaches the second surface 108b of the washer 108 to the exposed adhesive material on the first surface 106a of the adherent flange 106.

The user then inserts the proximal end 101a of the first flexible drain section 101 of the drainage tubing 104 into an incision site 110 of the patient under the skin surface 109 as illustrated in FIG. 1C and further into the wound, and attaches the first surface 108a of the washer 108 to the skin surface 109, for example, using surgical glue, thereby securing the surgical drain system 100 to the patient's body, free of sutures, leaving behind the proximal end 101a of the first flexible drain section 101 at a required location of the wound. The user squeezes the suction drainage container 105 to create a negative pressure therewithin and then connects the second end 102b of the second flexible drain section 102 of the drainage tubing 104 to the port 105a of the suction drainage container 105. The second end 102b of the second flexible drain section 102 of the drainage tubing 104 slips into the port 105a of the suction drainage container 105. The negative pressure within the suction drainage container 105 suctions the fluid from the wound at or proximal to the incision site 110 into the suction drainage container 105, thereby draining the fluid from the wound and allowing the wound to heal. The suctioned fluid collects in the suction drainage container 105. The user empties out the suction drainage container 105 at regular intervals.

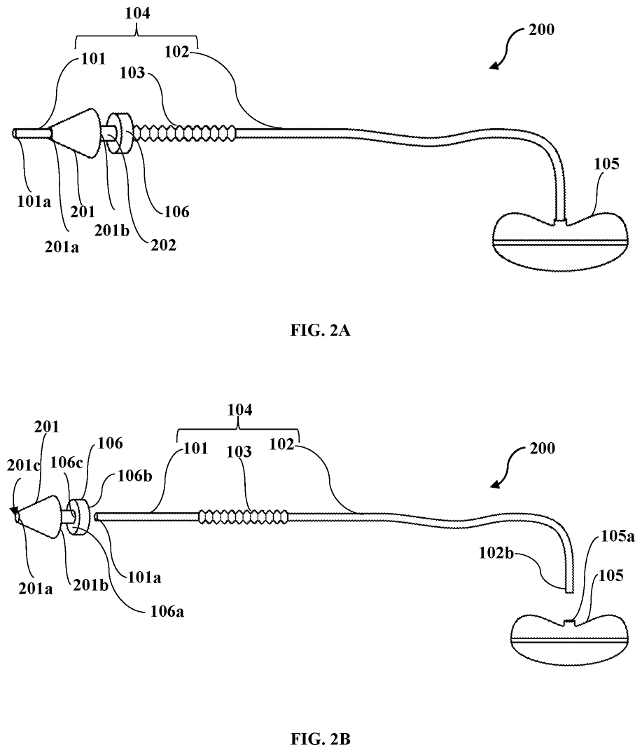

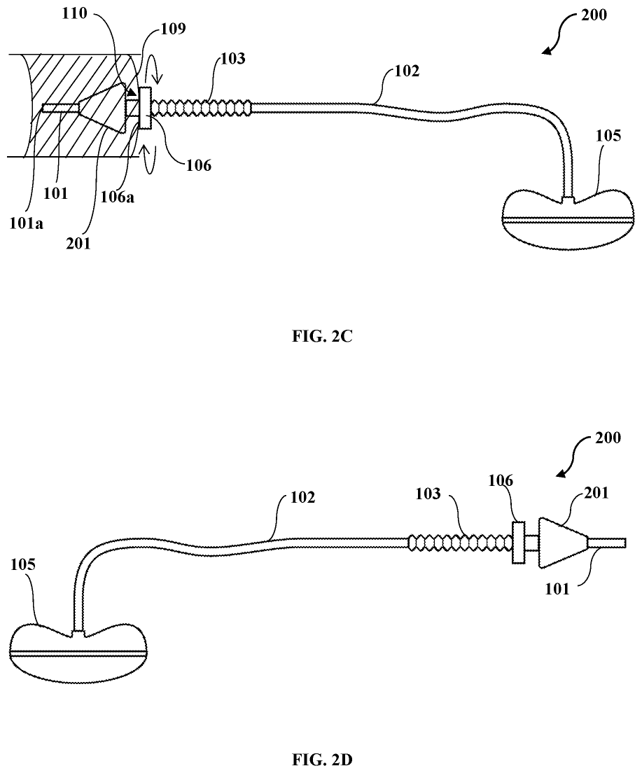

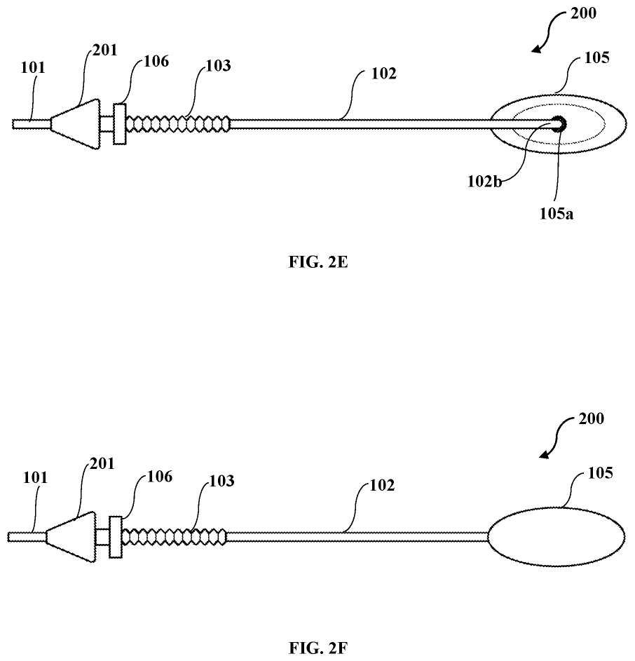

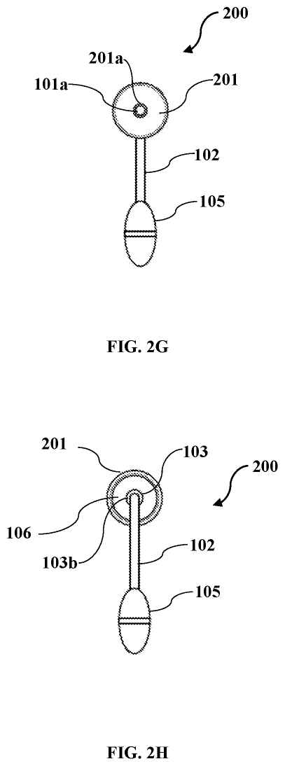

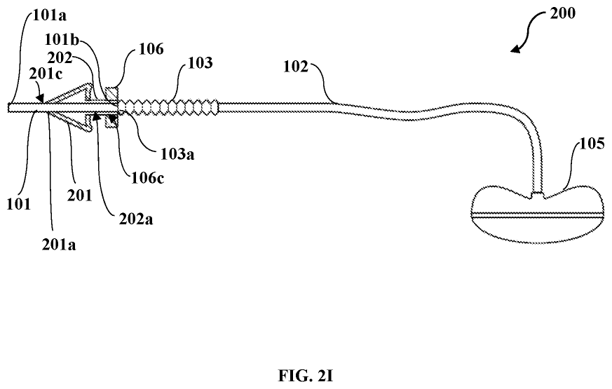

FIGS. 2A-2B illustrate assembled and disassembled, right-side perspective views of another embodiment of the surgical drain system 200. The surgical drain system 200 comprises an adherent flange 106, a drainage tubing 104, and a suction drainage container 105. The adherent flange 106 is configured to secure the surgical drain system 200 to a body of a patient, free of sutures. The adherent flange 106 comprises a first surface 106a, a second surface 106b, and a central opening 106c as illustrated in FIG. 2B. The adherent flange 106 is configured to be attached to a skin surface 109 of the body via the first surface 106a and be disposed external to an incision site 110 of the body as illustrated in FIG. 2C. In an embodiment, the first surface 106a of the adherent flange 106 is an adhesive surface configured to be attached directly to the skin surface 109. In another embodiment, the first surface 106a of the adherent flange 106 is a smooth surface configured to be attached to the skin surface 109 using surgical glue.

The drainage tubing 104 is operably coupled to the adherent flange 106. In an embodiment as illustrated in FIGS. 2A-2B, the drainage tubing 104 comprises a first flexible drain section 101, a flexible, cone-shaped, hollow member 201, a flexible corrugated section 103, and a second flexible drain section 102. The first flexible drain section 101 is inserted through the central opening 106c of the adherent flange 106 for insertion into the incision site 110. The first flexible drain section 101 comprises a proximal end 101a and a distal end 101b as illustrated in FIG. 2I. The proximal end 101a of the first flexible drain section 101 is configured to be inserted into the incision site 110 under the skin surface 109 and further into a wound or a surgical site (not shown) in the body to receive fluid from the wound or the surgical site. In an embodiment, the adherent flange 106 is movable over the first flexible drain section 101 to accommodate a required length of the first flexible drain section 101 during insertion into the incision site 110. In an embodiment, the drainage tubing 104 receives and passes the fluid from the wound or the surgical site into the suction drainage container 105 via the first flexible drain section 101. The fluid received through the first flexible drain section 101 passes through the drainage tubing 104 and is suctioned into and collected in the suction drainage container 105.

The flexible, cone-shaped, hollow member 201 is attached proximal to the proximal end 101a of the first flexible drain section 101. The flexible, cone-shaped, hollow member 201 is configured to be inserted into and disposed inside the incision site 110 under the skin surface 109 and to operate as a stopper to preclude the proximal end 101a of the first flexible drain section 101 from being accidentally dislodged from the incision site 110. The flexible, cone-shaped, hollow member 201 is made, for example, from silicone, latex, flexible rubber, etc. In an embodiment, the flexible, cone-shaped, hollow member 201 comprises a truncated end 201a and a base 201b as illustrated in FIGS. 2A-2B. The truncated end 201a of the flexible, cone-shaped, hollow member 201 comprises an opening 201c disposed coaxial to the central opening 106c of the adherent flange 106 as illustrated in FIG. 2B. The opening 201c of the truncated end 201a of the flexible, cone-shaped, hollow member 201 is attached proximal to the proximal end 101a of the first flexible drain section 101. The base 201b of the flexible, cone-shaped, hollow member 201 is configured to flexibly plug the first flexible drain section 101 against the skin surface 109 inside the body, after insertion of the flexible, cone-shaped, hollow member 201 with the proximal end 101a of the first flexible drain section 101 into the incision site 110. In an embodiment, the flexible, cone-shaped, hollow member 201 is connected to the adherent flange 106 via an extension 202 as illustrated in FIG. 2A.

The structure and the function of the flexible corrugated section 103 and the second flexible drain section 102 of the surgical drain system 200 are the same as those of the surgical drain system 100 disclosed in the description of FIGS. 1A-1B. The suction drainage container 105 is detachably connected to the second end 102b of the second flexible drain section 102 of the drainage tubing 104. The structure and the function of the suction drainage container 105 is the same as those of the surgical drain system 100 disclosed in the description of FIGS. 1A-1B.

FIGS. 2C-2D illustrate a right-side elevation view and a left-side elevation view of the embodiment of the surgical drain system 200 shown in FIG. 2A, respectively. FIG. 2C illustrates the proximal end 101a of the drainage tubing 104 of the surgical drain system 200 inserted into an incision site 110 of a patient's body under a skin surface 109. That is, the proximal end 101a of the first flexible drain section 101 along with the flexible, cone-shaped, hollow member 201 of the drainage tubing 104 is inserted into the incision site 110 of the patient's body as illustrated in FIG. 2C. FIG. 2C also illustrates the first surface 106a of the adherent flange 106 lying flush against the skin surface 109 of the patient's body, when the proximal end 101a of the first flexible drain section 101 along with the flexible, cone-shaped, hollow member 201 of the drainage tubing 104 is inserted into the incision site 110 of the patient's body. The adherent flange 106 is disposed external to the incision site 110. Also illustrated in FIG. 2C is the flexible, cone-shaped, hollow member 201 inserted into and disposed inside the incision site 110 under the skin surface 109. The flexible, cone-shaped, hollow member 201 operates as a stopper to preclude the proximal end 101a of the first flexible drain section 101 from being accidentally dislodged from the incision site 110.

In an embodiment as illustrated in FIG. 2C, the base 201b of the flexible, cone-shaped, hollow member 201 is configured to lie proximal to the skin surface 109 inside the body, after the insertion of the flexible, cone-shaped, hollow member 201 with the proximal end 101a of the first flexible drain section 101 into the incision site 110. In another embodiment (not shown), the base 201b of the flexible, cone-shaped, hollow member 201 is configured to lie flush against the skin surface 109 inside the body, after the insertion of the flexible, cone-shaped, hollow member 201 with the proximal end 101a of the first flexible drain section 101 into the incision site 110. In an embodiment, the adherent flange 106 is rigidly connected to the distal end 101b of the first flexible drain section 101, and when rotated, the adherent flange 106 is configured to twist and shrink the flexible, cone-shaped, hollow member 201 to allow removal of the flexible, cone-shaped, hollow member 201 and the first flexible drain section 101 from the incision site 110. The twisted flexible, cone-shaped, hollow member 201 allows convenient removal of the flexible, cone-shaped, hollow member 201 and the proximal end 101a of the first flexible drain section 101 from the incision site 110.

FIGS. 2E-2F illustrate a top plan view and a bottom elevation view of the embodiment of the surgical drain system 200 shown in FIG. 2A, respectively. The top plan view in FIG. 2E illustrates the second end 102b of the second flexible drain section 102 connected to the port 105a of the suction drainage container 105.

FIGS. 2G-2H illustrate a front elevation view and a rear elevation view of the embodiment of the surgical drain system 200 shown in FIG. 2A, respectively. The front elevation view in FIG. 2G illustrates the proximal end 101a of the first flexible drain section 101 extending through the truncated end 201a of the flexible, cone-shaped, hollow member 201 illustrated in FIG. 2B. The rear elevation view in FIG. 2H illustrates the second flexible drain section 102 extending from the second end 103b of the flexible corrugated section 103 of the drainage tubing 104 illustrated in FIGS. 2A-2B.

FIG. 2I illustrates a partial sectional view of the embodiment of the surgical drain system 200 shown in FIG. 2A. The partial sectional view in FIG. 2I illustrates the attachment of the truncated end 201a of the flexible, cone-shaped, hollow member 201 proximal to the proximal end 101a of the first flexible drain section 101. The partial sectional view in FIG. 2I also illustrates the first flexible drain section 101 passing through the central openings 106c, 202a, and 201c of the adherent flange 106, the extension 202, and the truncated end 201a of the flexible, cone-shaped, hollow member 201, respectively. As illustrated in FIG. 2I, the distal end 101b of the first flexible drain section 101 is connected to the first end 103a of the flexible corrugated section 103. In an embodiment, the base 201b of the flexible, cone-shaped, hollow member 201 is configured to slide on the first flexible drain section 101 between the skin surface 109 illustrated in FIG. 2C and the adherent flange 106.

Consider an example where a wound of a patient needs to be drained using the surgical drain system 200 comprising the adherent flange 106, the drainage tubing 104, and the suction drainage container 105 illustrated in FIGS. 2A-2I. A user, for example, a nurse practitioner, inserts the flexible, cone-shaped, hollow member 201 that is attached proximal to the proximal end 101a of the first flexible drain section 101 inside an incision site 110 of the patient under the skin surface 109, and attaches the first surface 106a of the adherent flange 106 to the skin surface 109 external to the incision site 110 as illustrated in FIG. 2C. The proximal end 101a of the first flexible drain section 101 that is disposed under the skin surface 109 is further positioned at or proximal to the wound for receiving fluid from the wound at or proximal to the incision site 110. The flexible, cone-shaped, hollow member 201 operates as a stopper and precludes the proximal end 101a of the first flexible drain section 101 from being accidentally dislodged from the incision site 110. The user squeezes the suction drainage container 105 to create a negative pressure therewithin and then connects the second end 102b of the second flexible drain section 102 of the drainage tubing 104 to the port 105a of the suction drainage container 105. The second end 102b of the second flexible drain section 102 of the drainage tubing 104 slips into the port 105a of the suction drainage container 105. The negative pressure within the suction drainage container 105 suctions the fluid from the wound at or proximal to the incision site 110 into the suction drainage container 105, thereby draining the fluid from the wound and allowing the wound to heal. The suctioned fluid collects in the suction drainage container 105. The user empties out the suction drainage container 105 at regular intervals.

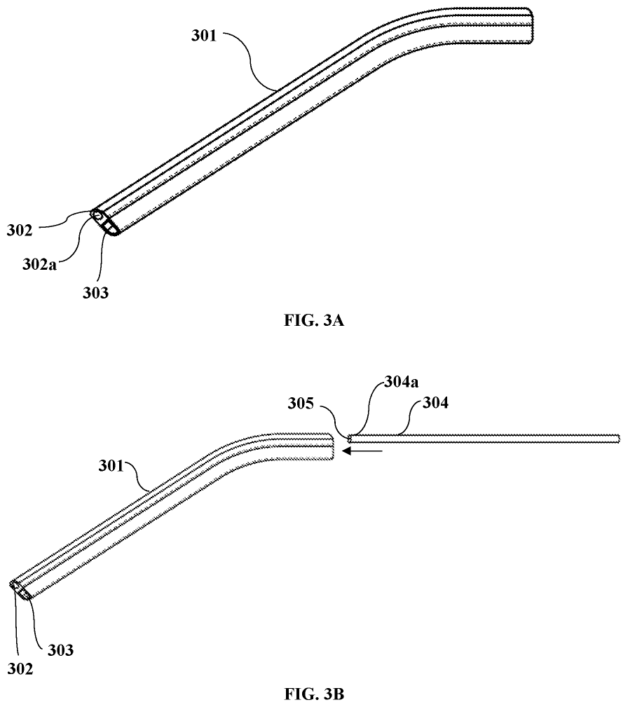

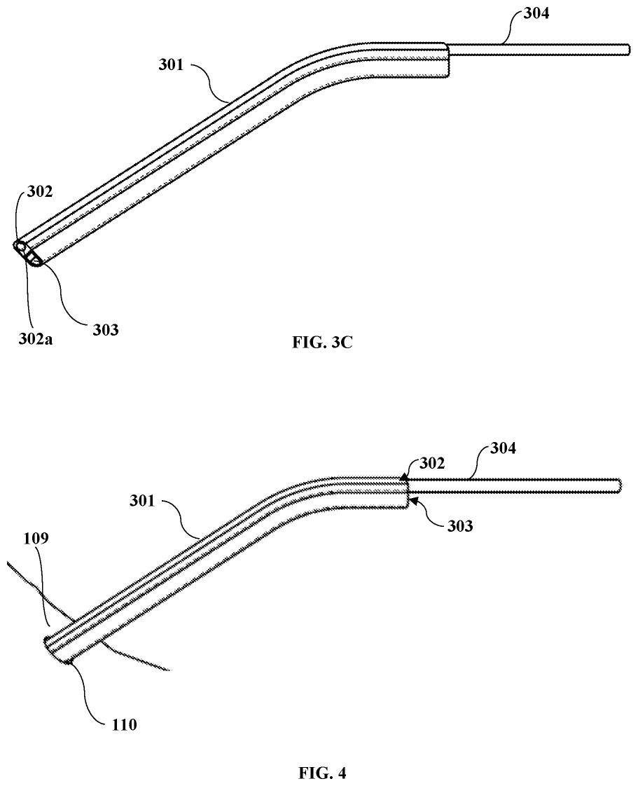

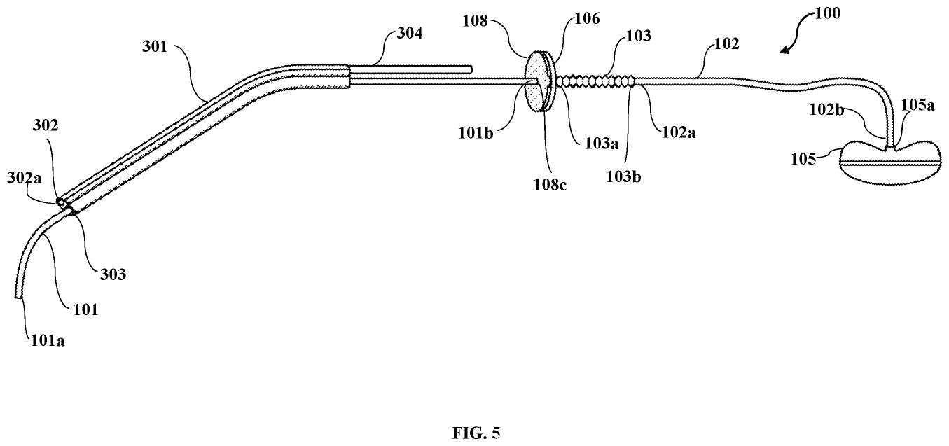

FIGS. 3A-3C illustrate right-side perspective views of an embodiment of a retractable sheath 301 configured to allow proper positioning of the proximal end 101a of the drainage tubing 104 of the surgical drain system 100 in an incision site 110 shown in FIG. 1C and FIG. 4. The retractable sheath 301 illustrated in FIG. 3A is configured to be inserted into and retracted from the incision site 110 illustrated in FIG. 4. In an embodiment as illustrated in FIG. 3B, an illumination element 304 with a camera 305 is provided to illuminate and allow viewing of a wound or a surgical site (not shown) inside a patient's body. The camera 305, for example, a miniaturized wireless camera, is disposed at a proximal end 304a of the illumination element 304. In an embodiment, the retractable sheath 301 comprises one or more channels, for example, 302 and 303, as illustrated in FIGS. 3A-3C. The channel 302 is configured to receive the illumination element 304 with the camera 305 as illustrated in FIG. 3C. The channel 303 is configured to receive the first flexible drain section 101 of the drainage tubing 104 of the surgical drain system 100 as illustrated in FIG. 5. The illumination element 304 with the camera 305 is inserted into the channel 302 as illustrated in FIGS. 3B-3C for emitting light and recording images of the wound or the surgical site at or proximal to the incision site 110 inside the body. In an embodiment, the illumination element 304 is an optical waveguide, for example, a non-fiber optic optical waveguide, configured to transmit light to the wound or the surgical site at or proximal to the incision site 110. In an embodiment, light filtering elements are operably coupled to one or more surfaces of the retractable sheath 301 for emitting light of one or more frequencies to enhance visualization of specific tissues at the wound or the surgical site. The retractable sheath 301 with the illumination element 304 and the camera 305 guides the first flexible drain section 101 into the incision site 110, and is thereafter retracted after proper placement of the first flexible drain section 101.

In an embodiment, the illumination element 304 is long and substantially rigid similar to that of a 1 millimeter (mm) copper wire. Similar to a copper wire, the illumination element 304 is also substantially flexible and may be bent to a desired shape prior to or after insertion of the illumination element 304 into the channel 302 of the retractable sheath 301. The retractable sheath 301 is adapted to also bend along with the illumination element 304 when the illumination element 304 is bent, after the illumination element 304 is inserted into the channel 302 of the retractable sheath 301. In an embodiment, the illumination element 304 is bent to a desired shape prior to the insertion of the illumination element 304 into the channel 302 of the retractable sheath 301, and the retractable sheath 301 takes the shape of the bent illumination element 304 after the bent illumination element 304 is inserted into the channel 302 of the retractable sheath 301. In an embodiment, the illumination element 304 is long and substantially rigid similar to an about 0.5 mm to about 1 mm copper wire. In another embodiment, the illumination element 304 is long and substantially rigid similar to a copper wire having a thickness of over 1 mm.

In an embodiment, a proximal end 302a of the channel 302 of the retractable sheath 301 is closed using a transparent cover (not shown). The transparent cover prevents fluid from entering the channel 302 while allowing the camera 305 to provide an uninterrupted view of the wound or the surgical site inside a patient's body, at or proximal to the incision site 110.

FIG. 4 illustrates a right-side perspective view showing positioning of the retractable sheath 301 in an incision site 110. The retractable sheath 301 with the illumination element 304 and the camera 305 accommodated therein is inserted into the incision site 110 under the skin surface 109 as illustrated in FIG. 4. The illumination element 304 with the camera 305 that is inserted into the channel 302 of the retractable sheath 301 allows a user, for example, a medical practitioner or a nurse practitioner, to position the proximal end 101a of the first flexible drain section 101 of the drainage tubing 104 of the surgical drain system 100 shown in FIGS. 1A-1B, at a required location of a wound or a surgical site (not shown).

FIG. 5 illustrates a right-side perspective view showing positioning of the proximal end 101a of the drainage tubing 104 of the surgical drain system 100 shown in FIGS. 1A-1B, through the retractable sheath 301. The retractable sheath 301 is configured to accommodate and hold the first flexible drain section 101 of the drainage tubing 104 and the illumination element 304 with the camera 305 together. The retractable sheath 301 with the accommodated illumination element 304 and the camera 305 is inserted and positioned in an incision site 110 in a patient's body as illustrated in FIG. 4. In an embodiment, the first flexible drain section 101 of the drainage tubing 104 is detached from the washer 108 and the adherent flange 106 and inserted into the channel 303 of the positioned retractable sheath 301 to allow the positioning of the proximal end 101a of the first flexible drain section 101 at a required location of a wound or a surgical site, at or proximal to the incision site 110, through the retractable sheath 301. The illumination element 304 with the camera 305 illuminates and allows viewing of the wound or the surgical site inside the body, After the proximal end 101a of the first flexible drain section 101 is inserted and positioned at the required location of the wound or surgical site, the retractable sheath 301 with the illumination element 304 and the camera 305 is retracted from the incision site 110. The distal end 101b of the first flexible drain section 101 is then inserted through the central openings 108c and 106c of the washer 108 and the adherent flange 106, respectively, illustrated in FIG. 1B, and connected to the first end 103a of the flexible corrugated section 103 of the drainage tubing 104. In an embodiment, the adherent flange 106 is movable to allow the adherent flange 106 to be moved and positioned over the first flexible drain section 101 to accommodate the required length of the first flexible drain section 101 after placement of the first flexible drain section 101 in the incision site 110. The second end 102b of the second flexible drain section 102 of the drainage tubing 104 is connected to the suction drainage container 105 as disclosed in the description of FIGS. 1A-1B.

Consider an example where a wound of a patient needs to be drained using the surgical drain system 100 illustrated in FIG. 5. In this example, the surgical drain system 100 in a disassembled state comprises the retractable sheath 301 with the illumination element 304 and the camera 305, the first flexible drain section 101, the washer 108, the adherent flange 106, the flexible corrugated section 103, the second flexible drain section 102, and the suction drainage container 105 as separate elements. A user, for example, a nurse practitioner, inserts the retractable sheath 301 with the illumination element 304 and the camera 305 into an incision site 110 as illustrated in FIG. 4. The user then passes the first flexible drain section 101 into the channel 303 of the retractable sheath 301 and into the incision site 110 under the skin surface 109. The user uses the illumination element 304 and the camera 305 to visualize and position the proximal end 101a of the first flexible drain section 101 at a required location of the wound. After positioning the proximal end 101a of the first flexible drain section 101 at the required location of the wound, the user retracts the retractable sheath 301 with the illumination element 304 and the camera 305, leaving behind the proximal end 101a of the first flexible drain section 101 at the required location of the wound. The user attaches the first surface 106a of the adherent flange 106 to the second surface 108b of the washer 108 illustrated in FIG. 1B. The user then inserts the distal end 101b of the first flexible drain section 101 through the central openings 108c and 106c of the washer 108 and the adherent flange 106, respectively, and slides the washer 108 and the adherent flange 106 over the first flexible drain section 101 to the incision site 110. The user adheres the first surface 108a of the washer 108 to the skin surface 109 at the incision site 110 as illustrated in FIG. 1C.

The user cuts the extended length of the first flexible drain section 101 and in an embodiment, fixes a mating connector, for example, a female connector (not shown), to the distal end 101b of the first flexible drain section 101, external to the incision site 110. In an embodiment, another mating connector, for example, a male connector (not shown), is operably coupled to the first end 103a of the flexible corrugated section 103. The mating connectors are similar to mating couplers or plugs that are used to connect flexible tubes in medical applications. The user connects the male connector at the first end 103a of the flexible corrugated section 103 to the female connector at the distal end 101b of the first flexible drain section 101 to connect the flexible corrugated section 103 to the first flexible drain section 101. The user then connects the first end 102a of the second flexible drain section 102 to the second end 103b of the flexible corrugated section 103. In an embodiment, the flexible corrugated section 103 and the second flexible drain section 102 are provided as a single continuous tube. The user squeezes the suction drainage container 105 to create a negative pressure therewithin and then connects the second end 102b of the second flexible drain section 102 to the port 105a of the suction drainage container 105. The negative pressure within the suction drainage container 105 suctions the fluid from the wound at or proximal to the incision site 110 into the suction drainage container 105, thereby draining the fluid from the wound and allowing the wound to heal. The suctioned fluid collects in the suction drainage container 105. The user empties out the suction drainage container 105 at regular intervals.

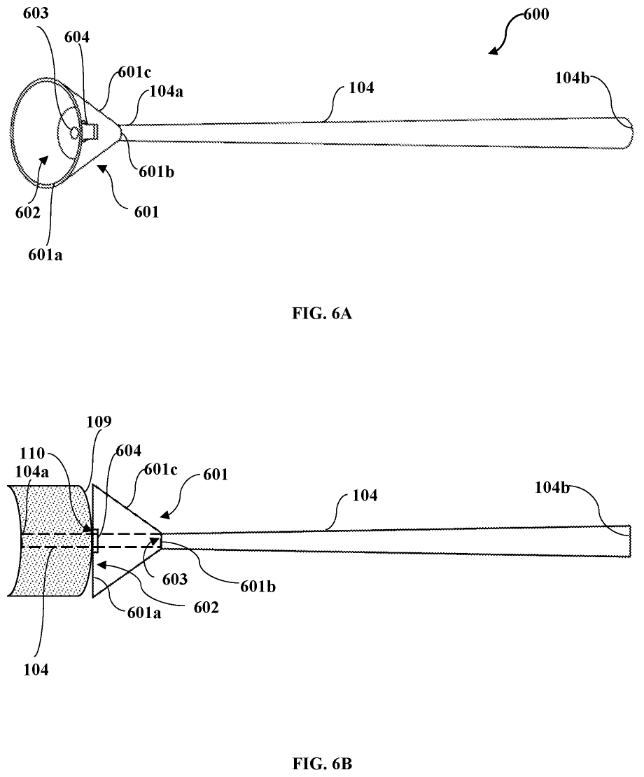

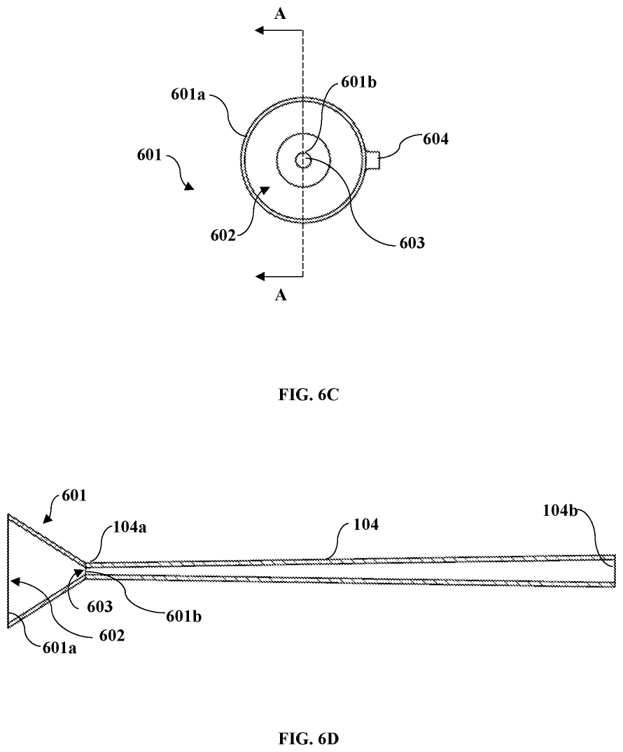

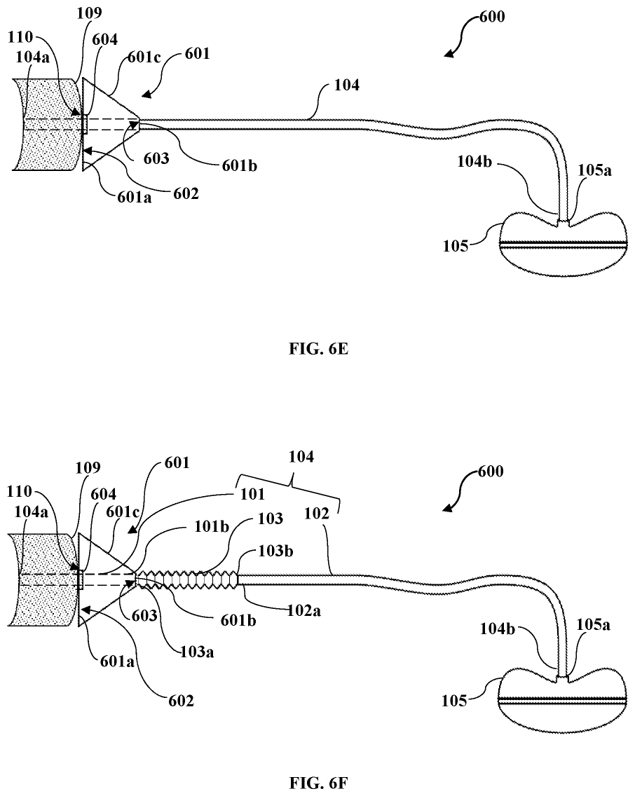

FIG. 6A illustrates an assembled, right-side perspective view of another embodiment of the surgical drain system 600. In this embodiment, the surgical drain system 600 comprises an adherent washer 601 and a drainage tubing 104 as illustrated in FIGS. 6A-6E. The adherent washer 601 is configured to secure the surgical drain system 600 to a patient's body, free of sutures. In an embodiment as illustrated in FIG. 6A, the adherent washer 601 is configured as a cone-shaped, hollow member comprising a curved wall 601c defined between a first end 601a and a second end 601b of the adherent washer 601. The first end 601a of the adherent washer 601 defines a first opening 602. The second end 601b of the adherent washer 601 defines a second opening 603. In an example, the height of the adherent washer 601 from the first end 601a to the second end 601b is about 0.5 inch to about 1 inch. In an embodiment as illustrated in FIG. 6A, the diameter of the first opening 602 is generally larger than the diameter of the second opening 603. In an example, the diameter of the first opening 602 is about 6 inches; and the diameter of the second opening 603 ranges from about 0.15 inches to about 0.3 inches. The adherent washer 601 is configured to be attached to a skin surface 109 of the body and be disposed external to an incision site 110 of the body as illustrated in FIG. 6B and as disclosed in the description of FIG. 6B. The adherent washer 601 is made, for example, from molded plastic, silicone, etc. In an embodiment, a tab 604 extends from the first end 601a of the adherent washer 601. The tab 604 is configured to allow removal of the surgical drain system 600 from the incision site 110.

The drainage tubing 104 of the surgical drain system 600 is operably coupled to the adherent washer 601. The drainage tubing 104 is a single, continuous tube having an outer diameter or a French size of, for example, about 3 Fr to about 10 Fr, that is, about 1 millimeter (mm) to about 3.33 mm. In another example, the adherent washer 601 is configured to accommodate drainage tubing 104 having diameters between about 1.33 mm to about 10.47 mm. The drainage tubing 104 is configured to pass through the second opening 603 and the first opening 602 of the adherent washer 601 into the incision site 110. The drainage tubing 104 comprises a proximal end 104a and a distal end 104b as illustrated in FIGS. 6A-6B. The proximal end 104a of the drainage tubing 104 is configured to be inserted into the incision site 110 under the skin surface 109 and further into a wound or a surgical site (not shown) in the body to receive fluid from the wound or the surgical site.

FIG. 6B illustrates a right-side elevation view of the embodiment of the surgical drain system 600 shown in FIG. 6A, showing the proximal end 104a of the drainage tubing 104 of the surgical drain system 600 inserted into an incision site 110 under a skin surface 109. The first end 601a of the adherent washer 601 is configured to be attached to the skin surface 109 of a patient's body and be disposed external to the incision site 110 of the body as illustrated in FIG. 6B. In an embodiment, the first end 601a of the adherent washer 601 is attached to the skin surface 109 using surgical glue. Examples of adhesives used on the first end 601a of the adherent washer 601 to attach the first end 601a of the adherent washer 601 to the skin surface 109 are removable medical adhesives, 3M® adhesives, etc. When attached, the first end 601a of the adherent washer 601 lies flush against the skin surface 109 as illustrated in FIG. 6B. The proximal end 104a of the drainage tubing 104 passes through the second opening 603 and the first opening 602 of the adherent washer 601 into the incision site 110. The proximal end 104a of the drainage tubing 104 is inserted into the incision site 110 under the skin surface 109 as illustrated in FIG. 6B and further into a wound or a surgical site (not shown) to receive fluid from the wound or the surgical site.

FIG. 6C illustrates a front elevation view of the embodiment of the surgical drain system 600 shown in FIG. 6A. The front elevation view in FIG. 6C illustrates the coaxial openings 602 and 603 and the tab 604 of the adherent washer 601. A user may hold the tab 604 between two fingers and pull out the drainage tubing 104 along with the adherent washer 601 from an incision site 110 illustrated in FIG. 6B. In an embodiment, the drainage tubing 104 may be removed from a patient's body after a predetermined time period, for example, about 14 days, while leaving the adherent washer 601 on the patient's skin surface 109 illustrated in FIG. 6B, until the adhesive or surgical glue comes off from the body. In an embodiment, when the drainage tubing 104 is removed from the body, the adherent washer 601 is attached to and left on the skin surface 109, for example, using an adhesive bandage, to cover the wound. The adhesive bandage is disposed on the adherent washer 601 externally on the skin surface 109.

FIG. 6D illustrates a cross-sectional view of the embodiment of the surgical drain system 600 shown in FIG. 6A, taken along a sectional line A-A shown in FIG. 6C. The cross-sectional view in FIG. 6D illustrates the proximal end 104a of the drainage tubing 104 disposed at the second end 601b of the adherent washer 601, prior to insertion into the openings 603 and 602 of the adherent washer 601, and thereafter into the incision site 110 under the skin surface 109 as illustrated in FIG. 6B, and further into the wound or the surgical site (not shown).

FIG. 6E illustrates a right-side elevation view of the embodiment of the surgical drain system 600 shown in FIG. 6A, showing the distal end 104b of the drainage tubing 104 of the surgical drain system 600 connected to a suction drainage container 105. The surgical drain system 600 further comprises a suction drainage container 105 detachably connected to the distal end 104b of the drainage tubing 104 as illustrated in FIG. 6E. The suction drainage container 105 is configured to create a negative pressure therewithin to suction and collect the fluid received from a wound or a surgical site (not shown) through the drainage tubing 104 as disclosed in the description of FIGS. 1A-1B. The suction drainage container 105 comprises a port 105a for inserting and connecting the distal end 104b of the drainage tubing 104 to the suction drainage container 105.

FIG. 6F illustrates a right-side elevation view of the embodiment of the surgical drain system 600 shown in FIG. 6A, showing another embodiment of the drainage tubing 104 of the surgical drain system 600. In this embodiment, the drainage tubing 104 is configured as a single continuous tube comprising three sections, that is, a first flexible drain section 101, a second flexible drain section 102, and a flexible corrugated section 103 as disclosed in the description of FIGS. 1A-1B. The first end 601a of the adherent washer 601 is attached to the skin surface 109 of a patient's body and is disposed external to the incision site 110 of the body as illustrated in FIG. 6F. The proximal end 104a of the first flexible drain section 101 of the drainage tubing 104 is inserted into the openings 603 and 602 of the adherent washer 601, and thereafter into the incision site 110 under the skin surface 109 and positioned further at a wound or a surgical site (not shown) in the patient's body to receive fluid, for example, wound exudate, blood cells, blood products, blood clots, wound debris, dead cells, pus, serous fluid, etc., from the wound or the surgical site. The flexible corrugated section 103 is in fluid communication with the first flexible drain section 101 of the drainage tubing 104. The first end 103a of the flexible corrugated section 103 is connected to the distal end 101b of the first flexible drain section 101. In an embodiment, the first end 103a of the flexible corrugated section 103 is connected to the second end 601b of the adherent washer 601. The first end 103a of the flexible corrugated section 103 is configured to lie flush against the second end 601b of the adherent washer 601. The flexible corrugated section 103 expands and contracts along with movement of the body, thereby reducing pain and discomfort experienced by the patient. The first end 102a of the second flexible drain section 102 of the drainage tubing 104 is connected to the second end 103b of the flexible corrugated section 103. The second flexible drain section 102 is in fluid communication with the flexible corrugated section 103. The suction drainage container 105 is detachably connected to the second end of the second flexible drain section 102, that is, to the distal end 104b of the drainage tubing 104. The fluid received through the first flexible drain section 101 of the drainage tubing 104 is suctioned into and collected in the suction drainage container 105 as disclosed in the description of FIGS. 1A-1B.

Consider an example where a wound of a patient needs to be drained using the surgical drain system 600 comprising the adherent washer 601, the drainage tubing 104, and the suction drainage container 105 illustrated in FIGS. 6A-6E. The drainage tubing 104 is operably coupled to the adherent washer 601 as illustrated in FIG. 6A. A user, for example, a nurse practitioner, slips the proximal end 104a of the drainage tubing 104 through the openings 603 and 602 of the adherent washer 601 and into an incision site 110 of the patient under the skin surface 109 as illustrated in FIG. 6B, and further into the wound, and attaches the first end 601a of the adherent washer 601 to the skin surface 109 of the patient's body using surgical glue, thereby securing the surgical drain system 600 to the patient's body, free of sutures, leaving behind the proximal end 104a of the drainage tubing 104 at the required location of the wound at or proximal to the incision site 110. In an embodiment, the user peels a peel and stick adhesive component provided on the first end 601a of the adherent washer 601 and attaches the exposed adhesive material on the first end 601a of the adherent washer 601 to the skin surface 109. The user squeezes the suction drainage container 105 to create a negative pressure therewithin and then connects the distal end 104b of the drainage tubing 104 to the port 105a of the suction drainage container 105 as illustrated in FIG. 6E. The distal end 104b of the drainage tubing 104 slips into the port 105a of the suction drainage container 105. The negative pressure within the suction drainage container 105 suctions the fluid from the wound at or proximal to the incision site 110 into the suction drainage container 105, thereby draining the fluid from the wound and allowing the wound to heal. The suctioned fluid collects in the suction drainage container 105. The user empties out the suction drainage container 105 at regular intervals.

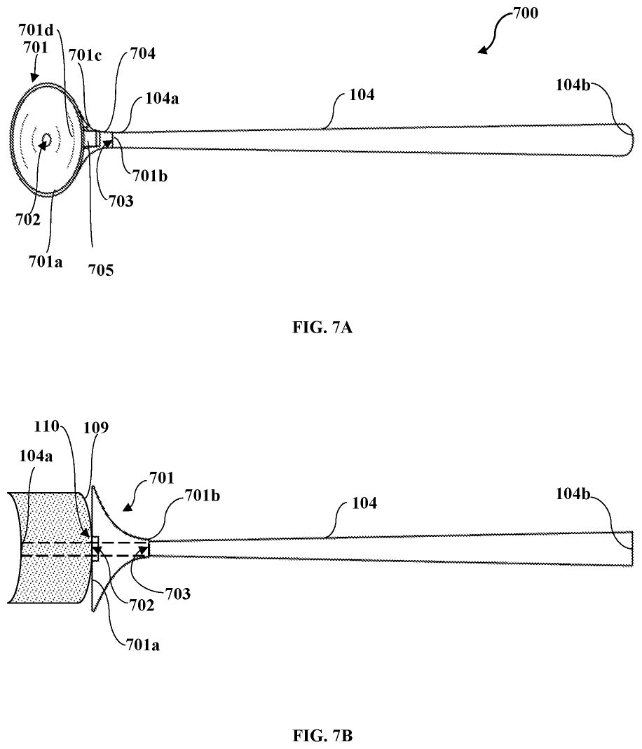

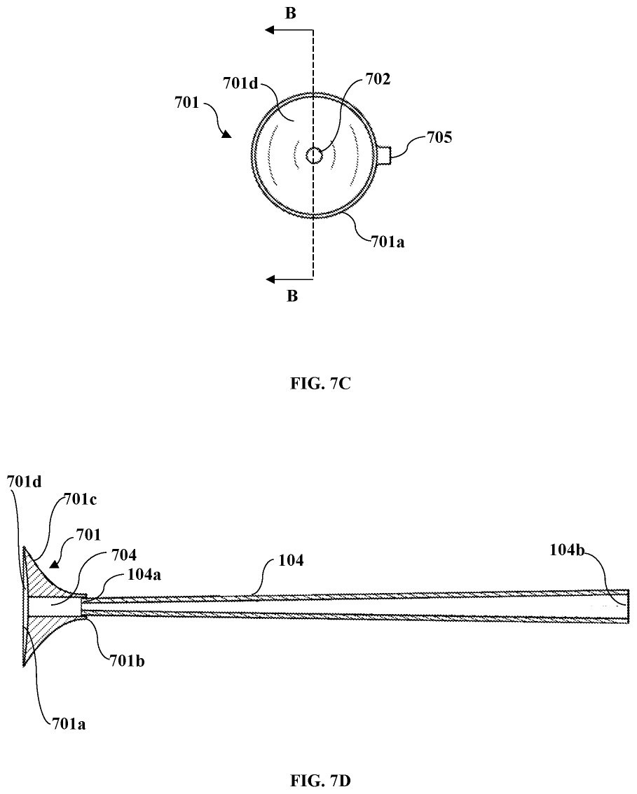

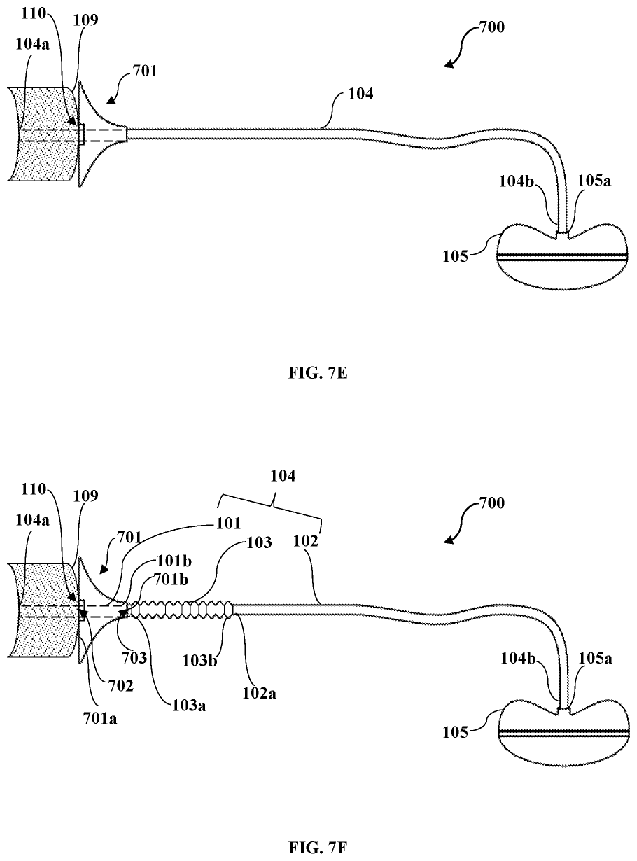

FIG. 7A illustrates an assembled, right-side perspective view of another embodiment of the surgical drain system 700. In this embodiment, the surgical drain system 700 comprises an adherent washer 701 and a drainage tubing 104 as illustrated in FIGS. 7A-7E. The adherent washer 701 is configured to secure the surgical drain system 700 to a patient's body, free of sutures. In an embodiment as illustrated in FIG. 7A, the adherent washer 701 comprises a partially concave surface 701d defined at a first end 701a of the adherent washer 701. The adherent washer 701 further comprises a curved wall 701c defined between the first end 701a and a second end 701b of the adherent washer 701. The partially concave surface 701d of the adherent washer 701 comprises a centrally located first opening 702. The second end 701b of the adherent washer 701 defines a second opening 703. In an example, the height of the adherent washer 701 from the first end 701a to the second end 701b is about 0.5 inch to about 1 inch. In an embodiment as illustrated in FIG. 7A, the diameter of the first opening 702 is the same as the diameter of the second opening 703 to allow the drainage tubing 104 to pass therethrough. In an example, the diameter of each of the first opening 702 and the second opening 703 ranges from about 0.13 inches to about 0.2 inches. The adherent washer 701 is configured to be attached to a skin surface 109 of the body and be disposed external to an incision site 110 of the body as illustrated in FIG. 7B and as disclosed in the description of FIG. 7B. The adherent washer 701 is made, for example, from molded plastic, silicone, etc. In an embodiment, a tab 705 extends from the first end 701a of the adherent washer 701. The tab 705 is configured to allow removal of the surgical drain system 700 from the incision site 110.

The drainage tubing 104 of the surgical drain system 700 is operably coupled to the adherent washer 701. The drainage tubing 104 is a single, continuous tube having an outer diameter or a French size of, for example, about 3 Fr to about 10 Fr, that is, about 1 millimeter (mm) to about 3.33 mm as disclosed in the description of FIG. 6A. The drainage tubing 104 is configured to pass through the second opening 703 and the first opening 702 of the adherent washer 701 into the incision site 110. The drainage tubing 104 comprises a proximal end 104a and a distal end 104b as illustrated in FIGS. 7A-7B. The proximal end 104a of the drainage tubing 104 is configured to be inserted into the incision site 110 under the skin surface 109 and further into a wound or a surgical site (not shown) in the body to receive fluid from the wound or the surgical site. In an embodiment, the adherent washer 701 further comprises a tubular holder 704 configured internally between the first end 701a and the second end 701b of the adherent washer 701, inside the curved wall 701c of the adherent washer 701 as illustrated in FIG. 7D. The tubular holder 704 extends internally from the first end 701a to the second end 701b of the adherent washer 701 as illustrated in FIG. 7D. The tubular holder 704 is configured to receive, stabilize, and pass the drainage tubing 104 into the incision site 110. The adherent washer 701 is slipped onto the drainage tubing 104 via the tubular holder 704 for stabilization of the drainage tubing 104.

FIG. 7B illustrates a right-side elevation view of the embodiment of the surgical drain system 700 shown in FIG. 7A, showing the proximal end 104a of the drainage tubing 104 of the surgical drain system 700 inserted into an incision site 110 under a skin surface 109. The first end 701a of the adherent washer 701 is configured to be attached to the skin surface 109 of a patient's body and be disposed external to the incision site 110 of the body as illustrated in FIG. 7B. In an embodiment, the first end 701a of the adherent washer 701 is attached to the skin surface 109 using surgical glue. Examples of adhesives used on the first end 701a of the adherent washer 701 to attach the first end 701a of the adherent washer 701 to the skin surface 109 are removable medical adhesives, 3M® adhesives, etc. The partially concave surface 701d of the adherent washer 701 that contacts the skin surface 109 is slightly scooped inward towards the first opening 702 as illustrated in FIG. 7A. When attached, the first end 701a of the adherent washer 701 lies flush against the skin surface 109 as illustrated in FIG. 7B. The proximal end 104a of the drainage tubing 104 passes through the second opening 703 and the first opening 702 of the adherent washer 701 into the incision site 110. The proximal end 104a of the drainage tubing 104 is inserted into the incision site 110 under the skin surface 109 as illustrated in FIG. 7B and further into a wound or a surgical site (not shown) to receive fluid from the wound or the surgical site.

FIG. 7C illustrates a front elevation view of the embodiment of the surgical drain system 700 shown in FIG. 7A. The front elevation view in FIG. 7C illustrates the first opening 702 and the tab 705 of the adherent washer 701. A user may hold the tab 705 between two fingers and pull out the drainage tubing 104 along with the adherent washer 701 from an incision site 110 illustrated in FIG. 7B. In an embodiment, the drainage tubing 104 may be removed from a patient's body after a predetermined time period, for example, about 14 days, while leaving the adherent washer 701 on the patient's skin surface 109 illustrated in FIG. 7B, until the adhesive or surgical glue comes off from the body. In an embodiment, when the drainage tubing 104 is removed from the body, the adherent washer 701 is attached to and left on the skin surface 109, for example, using an adhesive bandage, to cover the wound. The adhesive bandage is disposed on the adherent washer 701 externally on the skin surface 109.

FIG. 7D illustrates a cross-sectional view of the embodiment of the surgical drain system 700 shown in FIG. 7A, taken along a sectional line B-B shown in FIG. 7C. The cross-sectional view in FIG. 7D illustrates the proximal end 104a of the drainage tubing 104 disposed at the second end 701b of the adherent washer 701, prior to insertion into the openings 703 and 702 of the adherent washer 701, and thereafter into the incision site 110 under the skin surface 109 as illustrated in FIG. 7B, and further into the wound or the surgical site (not shown). The cross-sectional view in FIG. 7D also illustrates the tubular holder 704 extending from the first end 701a to the second end 701b of the adherent washer 701. The tubular holder 704 stabilizes the drainage tubing 104 during the insertion of the drainage tubing 104 into the openings 703 and 702 of the adherent washer 701, and thereafter into the incision site 110 under the skin surface 109, and further into the wound or the surgical site.

FIG. 7E illustrates a right-side elevation view of the embodiment of the surgical drain system 700 shown in FIG. 7A, showing the distal end 104b of the drainage tubing 104 of the surgical drain system 700 connected to a suction drainage container 105. The surgical drain system 700 further comprises a suction drainage container 105 detachably connected to the distal end 104b of the drainage tubing 104 as illustrated in FIG. 7E. The suction drainage container 105 is configured to create a negative pressure therewithin to suction and collect the fluid received from a wound or a surgical site (not shown) through the drainage tubing 104 as disclosed in the description of FIGS. 1A-1B. The suction drainage container 105 comprises a port 105a for inserting and connecting the distal end 104b of the drainage tubing 104 to the suction drainage container 105.

FIG. 7F illustrates a right-side elevation view of the embodiment of the surgical drain system 700 shown in FIG. 7A, showing another embodiment of the drainage tubing 104 of the surgical drain system 700. In this embodiment, the drainage tubing 104 is configured as a single continuous tube comprising three sections, that is, a first flexible drain section 101, a second flexible drain section 102, and a flexible corrugated section 103 as disclosed in the description of FIGS. 1A-1B. The first end 701a of the adherent washer 701 is attached to the skin surface 109 of a patient's body and is disposed external to the incision site 110 of the body as illustrated in FIG. 7F. The proximal end 104a of the first flexible drain section 101 of the drainage tubing 104 is inserted into the openings 703 and 702 of the adherent washer 701, and thereafter into the incision site 110 under the skin surface 109 and positioned further at a wound or a surgical site (not shown) in the patient's body to receive fluid, for example, wound exudate, blood cells, blood products, blood clots, wound debris, dead cells, pus, serous fluid, etc., from the wound or the surgical site. The flexible corrugated section 103 is in fluid communication with the first flexible drain section 101 of the drainage tubing 104. The first end 103a of the flexible corrugated section 103 is connected to the distal end 101b of the first flexible drain section 101. In an embodiment, the first end 103a of the flexible corrugated section 103 is connected to the second end 701b of the adherent washer 701. The first end 103a of the flexible corrugated section 103 is configured to lie flush against the second end 701b of the adherent washer 701. The flexible corrugated section 103 expands and contracts along with movement of the body, thereby reducing pain and discomfort experienced by the patient. The first end 102a of the second flexible drain section 102 is connected to the second end 103b of the flexible corrugated section 103. The second flexible drain section 102 is in fluid communication with the flexible corrugated section 103. The suction drainage container 105 is detachably connected to the second end of the second flexible drain section 102, that is, to the distal end 104b of the drainage tubing 104. The fluid received through the first flexible drain section 101 of the drainage tubing 104 is suctioned into and collected in the suction drainage container 105 as disclosed in the description of FIGS. 1A-1B.

Consider an example where a wound of a patient needs to be drained using the surgical drain system 700 comprising the adherent washer 701, the drainage tubing 104, and the suction drainage container 105 illustrated in FIGS. 7A-7E. The drainage tubing 104 is operably coupled to the adherent washer 701 as illustrated in FIG. 7A. A user, for example, a nurse practitioner, slips the proximal end 104a of the drainage tubing 104 through the openings 703 and 702 of the adherent washer 701 and into an incision site 110 of the patient under the skin surface 109 as illustrated in FIG. 7B, and further into the wound, and attaches the first end 701a of the adherent washer 701 to the skin surface 109 of the patient's body using surgical glue, thereby securing the surgical drain system 700 to the patient's body, free of sutures, leaving behind the proximal end 104a of the drainage tubing 104 at the required location of the wound at or proximal to the incision site 110. In an embodiment, the user peels a peel and stick adhesive component provided on the first end 701a of the adherent washer 701 and attaches the exposed adhesive material on the first end 701a of the adherent washer 701 to the skin surface 109. The user squeezes the suction drainage container 105 to create a negative pressure therewithin and then connects the distal end 104b of the drainage tubing 104 to the port 105a of the suction drainage container 105 as illustrated in FIG. 7E. The distal end 104b of the drainage tubing 104 slips into the port 105a of the suction drainage container 105. The negative pressure within the suction drainage container 105 suctions the fluid from the wound at or proximal to the incision site 110 into the suction drainage container 105, thereby draining the fluid from the wound and allowing the wound to heal. The suctioned fluid collects in the suction drainage container 105. The user empties out the suction drainage container 105 at regular intervals.

In an embodiment, the surgical drain systems 100, 200, 600, and 700 illustrated in FIGS. 1A-1H, FIGS. 2A-2I, FIGS. 6A-6F, and FIGS. 7A-7F, respectively, are made from materials that reduce irritation to a patient's skin and other complications. The first flexible drain section 101, the flexible, cone-shaped, hollow member 201, and the drainage tubing 104 are constructed from any material suitable for implantation with the patient's body and that reduces the chances of allergy or other reactions. Examples of materials for constructing the first flexible drain section 101 and the flexible, cone-shaped, hollow member 201 are biocompatible silicone, polyether ether ketone (PEEK), polyvinyl chloride (PVC), polytetrafluoroethylene (PTFE), etc. The drainage tubing 104 comprising the first flexible drain section 101, the flexible corrugated section 103, and the second flexible drain section 102 or the single continuous drainage tubing 104 illustrated in FIGS. 6A-6B and FIGS. 7A-7B, is constructed, for example, from PVC, PEEK, or a thermoplastic elastomer (TPE) such as a polyether block amine (PEBA). In an embodiment, a hydrophilic or lubricious coating is applied on the first flexible drain section 101 to assist in removal of the first flexible drain section 101 from the incision site 110. In another embodiment, a hydrophilic or lubricious coating is applied on inner and outer surfaces of the drainage tubing 104. The coating on the inner surfaces of the drainage tubing 104 is configured to reduce drag and shear force coefficients for improved flow of the fluid from the wound or the surgical site (not shown) into the drainage tubing 104. The coating on the outer surfaces of the drainage tubing 104 is configured to reduce friction upon removal of the drainage tubing 104 from the patient's body. In another embodiment, the drainage tubing 104 is extruded with antimicrobial agents to reduce the likelihood or effect of seroma and incision site infections. In another embodiment, the antimicrobial agents are applied to the drainage tubing 104 during or after manufacture of the drainage tubing 104.

The suture-less attachment of the drainage tubing 104 of the surgical drain system 100/200/600/700 to the skin surface 109 of the patient's body at the incision site 110 reduces pain experienced by the patient during insertion of the drainage tubing 104 into the incision site 110 and removal of the drainage tubing 104 out from the incision site 110. The adherent flange 106 of the surgical drain system 100/200 illustrated in FIGS. 1A-1H and FIGS. 2A-2I is used to attach the proximal end 101a of the first flexible drain section 101 to the incision site 110, instead of having the first flexible drain section 101 sutured on the skin surface 109 on both sides of the first flexible drain section 101, to reduce pain caused by the sutures on the skin surface 109. Similarly, the adherent washer 601/701 of the surgical drain system 600/700 illustrated in FIGS. 6A-6F and FIGS. 7A-7F is used to attach the proximal end 104a of the drainage tubing 104 to the incision site 110, instead of having the drainage tubing 104 sutured on the skin surface 109 on both sides of the drainage tubing 104, to reduce pain caused by the sutures on the skin surface 109. The flexible corrugated section 103 of the surgical drain system 100/200/600/700 illustrated in FIGS. 1A-1H, FIGS. 2A-2I, FIG. 6F, and FIG. 7F connects internal tubing, that is, the first flexible drain section 101 inside the skin surface 109, and external tubing, that is, the second flexible drain section 102 outside the skin surface 109 to further reduce pain to the patient due to the movement of the drainage tubing 104.

The foregoing examples and illustrative implementations of various embodiments have been provided merely for explanation and are in no way to be construed as limiting the embodiments disclosed herein. Dimensions of various parts of the surgical drain system disclosed above are exemplary, and are not limiting of the scope of the embodiments herein. While the embodiments have been described with reference to various illustrative implementations, drawings, and techniques, it is understood that the words, which have been used herein, are words of description and illustration, rather than words of limitation. Furthermore, although the embodiments have been described herein with reference to particular means, materials, techniques, and implementations, the embodiments herein are not intended to be limited to the particulars disclosed herein; rather, the embodiments extend to all functionally equivalent structures, methods and uses, such as are within the scope of the appended claims. It will be understood by those skilled in the art, having the benefit of the teachings of this specification, that the embodiments disclosed herein are capable of modifications and other embodiments may be effected and changes may be made thereto, without departing from the scope and spirit of the embodiments disclosed herein.

We claim:

1. A surgical drain system comprising:
an adherent flange configured to secure the surgical drain system to a body of a patient, free of sutures, via a washer, the adherent flange comprising a first surface, a second surface, and a central opening, wherein the adherent flange is configured to be attached to a skin surface of the body via the washer and be disposed external to an incision site of the body;
the washer detachably attachable and disposed coaxial to the adherent flange between the skin surface and the adherent flange;
a drainage tubing operably coupled to the adherent flange and the washer, the drainage tubing comprising:
a first flexible drain section inserted through the central opening of the adherent flange and the washer, the first flexible drain section comprising a proximal end and a distal end, wherein the proximal end of the first flexible drain section is configured to be inserted into the incision site under the skin surface and further into one of a wound and a surgical site in the body to receive fluid from the one of the wound and the surgical site;
a flexible corrugated section comprising a first end and a second end, wherein the first end of the flexible corrugated section is connected to the distal end of the first flexible drain section and is disposed external to the incision site, and wherein the flexible corrugated section is in fluid communication with the first flexible drain section; and
a second flexible drain section comprising a first end and a second end, wherein the first end of the second flexible drain section is connected to the second end of the flexible corrugated section, and wherein the second flexible drain section is in fluid communication with the flexible corrugated section; and
a suction drainage container detachably connected to the second end of the second flexible drain section of the drainage tubing, wherein the suction drainage container is configured to create a negative pressure therewithin to suction and collect the fluid received from the one of the wound and the surgical site through the drainage tubing.

2. The surgical drain system of claim 1, wherein the washer comprises a first surface, a second surface, and a central opening, wherein the first surface of the washer is configured to be attached flush against the skin surface, wherein the first surface of the adherent flange is configured to be attached flush against the second surface of the washer, and wherein the first flexible drain section of the drainage tubing is inserted through the central opening of each of the adherent flange and the washer for insertion into the incision site.

3. The surgical drain system of claim 2, wherein the first surface of the washer is an adhesive surface configured to be attached directly to the skin surface.

4. The surgical drain system of claim 2, wherein the first surface of the washer is configured to be attached to the skin surface using surgical glue.

5. The surgical drain system of claim 1, further comprising a tab extending from a periphery of the washer, wherein the tab is configured to allow removal of the surgical drain system from the incision site.

6. The surgical drain system of claim 1, wherein the first end of the flexible corrugated section of the drainage tubing is configured to lie flush against the second surface of the adherent flange, and wherein the flexible corrugated section is configured to reduce pain and discomfort experienced by the patient due to accidental movement of the first flexible drain section and the second flexible drain section of the drainage tubing.

7. The surgical drain system of claim 1, further comprising a flexible, cone-shaped, hollow member attached proximal to the proximal end of the first flexible drain section of the drainage tubing, wherein the flexible, cone-shaped, hollow member is configured to be inserted into and disposed inside the incision site under the skin surface and to operate as a stopper to preclude the proximal end of the first flexible drain section from being accidentally dislodged from the incision site.

8. The surgical drain system of claim 7, wherein the flexible, cone-shaped, hollow member comprises a truncated end and a base, wherein the truncated end comprises an opening disposed coaxial to the central opening of the adherent flange, wherein the opening of the truncated end of the flexible, cone-shaped, hollow member is attached proximal to the proximal end of the first flexible drain section of the drainage tubing, and wherein the base of the flexible, cone-shaped, hollow member is configured to flexibly plug the first flexible drain section against the skin surface inside the body, after insertion of the flexible, cone-shaped, hollow member with the proximal end of the first flexible drain section into the incision site.

9. The surgical drain system of claim 7, wherein the adherent flange is rigidly connected to the distal end of the first flexible drain section of the drainage tubing, and when rotated, the adherent flange is configured to twist and shrink the flexible, cone-shaped, hollow member to allow removal of the flexible, cone-shaped, hollow member and the first flexible drain section from the incision site.

10. The surgical drain system of claim 1, wherein the adherent flange is movable over the first flexible drain section of the drainage tubing to accommodate a required length of the first flexible drain section during insertion into the incision site.

11. The surgical drain system of claim 1, further comprising:
an illumination element with a camera configured to illuminate and allow viewing of the one of the wound and the surgical site inside the body; and
a retractable sheath configured to be inserted into and retracted from the incision site, wherein the retractable sheath comprises one or more channels configured to receive the illumination element with the camera and the first flexible drain section of the drainage tubing of the surgical drain system, wherein the illumination element with the camera allows a user to position the proximal end of the first flexible drain section at a required location of the one of the wound and the surgical site.

12. A surgical drain system comprising:
an adherent flange configured to secure the surgical drain system to a body of a patient, free of sutures, the adherent flange comprising a first surface, a second surface, and a central opening, wherein the adherent flange is configured to be attached to a skin surface of the body via the first surface and be disposed external to an incision site of the body;
a drainage tubing operably coupled to the adherent flange, the drainage tubing comprising:
a first flexible drain section inserted through the central opening of the adherent flange, the first flexible drain section comprising a proximal end and a distal end, wherein the proximal end of the first flexible drain section is configured to be inserted into the incision site under the skin surface and further into one of a wound and a surgical site in the body to receive fluid from the one of the wound and the surgical site;
a flexible, cone-shaped, hollow member attached proximal to the proximal end of the first flexible drain section, wherein the flexible, cone-shaped, hollow member is configured to be inserted into and disposed inside the incision site under the skin surface and to operate as a stopper to preclude the proximal end of the first flexible drain section from being accidentally dislodged from the incision site;
a flexible corrugated section comprising a first end and a second end, wherein the first end of the flexible corrugated section is connected to the distal end of the first flexible drain section and is disposed external to the incision site, and wherein the flexible corrugated section is in fluid communication with the first flexible drain section; and
a second flexible drain section comprising a first end and a second end, wherein the first end of the second flexible drain section is connected to the second end of the flexible corrugated section, and wherein the second flexible drain section is in fluid communication with the flexible corrugated section; and
a suction drainage container detachably connected to the second end of the second flexible drain section of the drainage tubing, wherein the suction drainage container is configured to create a negative pressure therewithin to suction and collect the fluid received from the one of the wound and the surgical site through the drainage tubing.

13. The surgical drain system of claim 12, wherein the flexible, cone-shaped, hollow member comprises a truncated end and a base, wherein the truncated end comprises an opening disposed coaxial to the central opening of the adherent flange, wherein the opening of the truncated end of the flexible, cone-shaped, hollow member is attached proximal to the proximal end of the first flexible drain section of the drainage tubing, and wherein the base of the flexible, cone-shaped, hollow member is configured to flexibly plug the first flexible drain section against the skin surface inside the body, after insertion of the flexible, cone-shaped, hollow member with the proximal end of the first flexible drain section into the incision site.

14. The surgical drain system of claim 13, wherein the base of the flexible, cone-shaped, hollow member is configured to slide on the first flexible drain section of the drainage tubing between the skin surface and the adherent flange.

15. The surgical drain system of claim 12, wherein the adherent flange is rigidly connected to the distal end of the first flexible drain section of the drainage tubing, and when rotated, the adherent flange is configured to twist and shrink the flexible, cone-shaped, hollow member to allow removal of the flexible, cone-shaped, hollow member and the first flexible drain section from the incision site.

16. The surgical drain system of claim 12, wherein the first end of the flexible corrugated section of the drainage tubing is configured to lie flush against the second surface of the adherent flange, and wherein the flexible corrugated section is configured to reduce pain and discomfort experienced by the patient due to accidental movement of the first flexible drain section and the second flexible drain section of the drainage tubing.

17. A surgical drain system comprising:
an adherent washer configured to secure the surgical drain system to a body of a patient, free of sutures, the adherent washer comprising a curved wall defined between a first end and a second end of the adherent washer, wherein the first end defines a first opening and the second end defines a second opening, and wherein the first end of the adherent washer is configured to be attached to a skin surface of the body and be disposed external to an incision site of the body;
a drainage tubing operably coupled to the adherent washer, wherein the drainage tubing is configured to pass through the second opening and the first opening of the adherent washer into the incision site, wherein the drainage tubing comprises a proximal end and a distal end, wherein the proximal end of the drainage tubing is configured to be inserted into the incision site under the skin surface and further into one of a wound and a surgical site in the body to receive fluid from the one of the wound and the surgical site; and
a suction drainage container detachably connected to the distal end of the drainage tubing, wherein the suction drainage container is configured to create a negative pressure therewithin to suction and collect the fluid received from the one of the wound and the surgical site through the drainage tubing.

18. The surgical drain system of claim 17, wherein the first end of the adherent washer is configured to be attached to the skin surface using surgical glue.

19. The surgical drain system of claim 17, wherein the adherent washer further comprises a tubular holder configured internally between the first end and the second end of the adherent washer, wherein the tubular holder is configured to receive, stabilize, and pass the drainage tubing into the incision site.

20. The surgical drain system of claim 17, further comprising a tab extending from the first end of the adherent washer, wherein the tab is configured to allow removal of the surgical drain system from the incision site.
